(19) United States
(12) Patent Application Publication
(10) Pub. No.: US 2009/0123377 A1
(43) Pub. Date: May 14, 2009

(54) Method for Testing Substances or Mixtures of Substances,Use of Said Method and Corresponding Test Kits

(75) Inventors: Elard Jacob, Eisenberg (DE); Herbert Platsch, Mannheim (DE); Gerhard Krennrich, Frankenthal (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, Ludwigshafen (DE)

(21) Appl. No.: 11/921,782

(22) PCT Filed: Jun. 8, 2006
(86) PCT No.: PCT/EP2006/005508

§ 371 Date: Dec. 7, 2007

(30) Foreign Application Priority Data
Jun. 9, 2005 (DE) 10 2005 026 710.6

(51) Int. Cl. A61K 49/00; G01N 33/53; A61P 43/00

(52) U.S. Cl. 424/9.2; 435/7.2

(57) ABSTRACT

The invention relates to a method for testing substances and mixtures of substances for toxic characteristics, to the use of said method and to corresponding test kits. The method is essentially based on the determination of a fatty acid binding protein from the liver, L-FABP. The use of said method provides an early indication of carcinogenic and tumour-promoting characteristics of the tested substance or mixture of substances.

The present invention relates to a method for testing substances and substance mixtures for toxic properties, the use thereof and corresponding analytical kits. The method is based substantially on determining a liver fatty acid binding protein (abbreviated to L-FABP). Application of this method provides in particular early indications of carcinogenic and, in particular, tumor-promoting properties of the tested substance or tested substance mixture.

A possible carcinogenic potential may represent a knockout criterion for the development of an active ingredient. Whereas such an effect is acceptable for active pharmaceutical ingredients, depending on the area of application, it is a serious hazard for the development of crop protection active ingredients. The principle applying in such cases is to differentiate between a carcinogenic potential attributable to a genotoxic effect, and a carcinogenic potential based on a non-genotoxic, tumor-promoting mechanism. In the latter case, an action threshold can be assumed and frequently also demonstrated experimentally, and the substance may be approved. It can generally be stated that genotoxic and non-genotoxic carcinogens differ in substantial points, amounting to differentiation between incomplete and complete carcinogens. Thus, effects are frequently reversible in the early promotion phase, and a tumor marker (genotoxic effect) is not per se a marker for tumor promotion (non-genotoxic effect). Moreover, according to the two-stage model of carcinogenesis, it is usually necessary for a tumor-promoting effect to be preceded by initiation of the affected cells.

The earliest possible information about a tumor-promoting property is important because conventional testing in a cancer-induction study is time consuming and costly. The investigations carried out into mutagenicity/genotoxicity do not result in a clear warning especially when genotoxic properties are absent or only weak at the same time. Tumors appear as a result of tumor-promoting properties only late in the study and with high dosages and their appearance is therefore difficult to categorize in terms of human relevance.

Tumor-promoting substances are particularly well described for the rodent liver (especially in rats). The liver as target organ is a good model system because most carcinogenic substances produce liver tumors in rodents, consistent with the primary role of the liver as main organ of detoxication. There is a broad mechanistic database on liver carcinogenicity. A number of groups of substances has been described among the non-genotoxic tumor-promoting substances with liver as target organ, and they include in particular those with receptor-mediated mechanisms of action, e.g. lipid-lowering agents and peroxisome proliferators, TCDD and analogs, and estrogen-like substances, enzyme inducers, e.g. DDT, alpha-hexachlorocyclohexane, phenobarbital, various crop protection agents and drugs, and AH receptor agonists and cytotoxic mitogenic substances, e.g. tetrachloromethane and tetrahydrofuran, substances which promote oxidative stress, e.g. FeNTA and substances with mitochondrial toxicity, e.g. some aromatic compounds, amines and furans.

Peroxisome proliferators are probably significant only for the rodent system. Functional test systems have already been developed for antihormonal substances and substances having a hormone-like effect on the male and female reproductive system. The manifestation in rodents is a significant enlargement of the liver, formation of preneoplastic, immunocytochemically detectable lesions which may develop into liver tumors in later phases. Various medium-term assays are available for detecting the preneoplastic lesions in animal experiments (e.g. initiation-promotion model of Ito (Ito. N.; Imaida, K.; Hasegawa, R.; Tsuda, H. Crit. Rev. Toxicol. (1989) 19, 385-415) or Schulte-Hermann (Schulte-Hermann, R.; Bursch, W.; Low-Baselli, A.; Wagner, A.; Grasl-Kraupp, B. Cell Biol. Toxicol. (1997) 13, 339-348). However, these are too elaborate for screening purposes. Screening systems based on transcriptome and proteome analyses are also associated with problems because there are usually changes in a large number of markers, and it is possible only with difficulty to distinguish between adaptive homeostatic and significantly (irreversibly) changed markers.

L-FABP is a commonly occurring protein in the rat liver, with a proportion of 2 to 6% of total cytosolic protein (S. Sorof, Cancer and Metastasis Reviews (1994) 13, 317-336). In total, seven fatty acid binding proteins are known and are named after the tissue from which they were originally isolated (R. Das, R. Hammamieh, R. Neill, M. Melhem, M. Jett, Clin. Cancer Res. (2001) 7, 1706-1715): a) adipocyte (A-FABP), b) heart or muscle (H-FABP), c) brain (B-FABP), d) epidermis or psoriasis-associated (E-FABP), e) liver (L-FABP), f) small intestine (1-FABP) and g) myelin or P2 (P2-FABP). The main task of FABPs is to transport long-chain fatty acids and other hydrophobic ligands, some of which are involved in signal transduction chains. Absence of certain fatty acid binding proteins or dysfunction thereof is suggested in the literature to be associated with dieases such as diabetes, hyperlipidemia, obesity, atherosclerosis and myocardial hypertrophy (J. F. Glatz, J. Storch, Curr. Opinion in Lipidology (2001) 12, 267-274).

There are indications that fatty acid binding proteins may have a function in cell division and differentiation. Schroeder et al. were able to show that L-FABP influences the growth and differentiation of embryonic stem cells (F. Schroeder, B P. Atshaves, O. Starodub, A. L. Boedeker, R. R. Smith III. J. B. Roths, W. B. Foxworth, A. B. Kier, Molecular and Cellular Biochemistry (2001) 219, 127-138). Sorof describes the modulation of mitogenesis by L-FABP (Sorof et al. 1994), supra). There is accordingly said to be a synergism between the effect of L-FABP and unsaturated fatty acids in promoting cellular proliferation. L-FABP is further said to be required to induce mitogenesis—induced by various classes of non-genotoxic hepatocarcinogenic peroxisome proliferators. Together with other indications, this is said to suggest that L-FABP is involved in regulating cell division in hepatocytes. Sorof et al. additionally described L-FABP as the polypeptide target of a genotoxic carcinogen (2-acetylaminofluorene) in rat hepatocytes (J. A. Bassuk, P. N. Tsichlis, S. Sorof, Proc. Nat. Acad. Sci. USA (1997) 84, 7547-7551). However, a very large increase in the protein in rat hepatocytes during the proliferation after induction by the carcinogen is also described there. This is consistent with Das et al, who found a marked upregulation (5-9-fold) of L- and I-FABP when comparing normal human prostate cells with corresponding cancer cells. A- and E-FABP were by contrast markedly (3-20-fold) downregulated in the cancer cells. These authors proposed FABPs as potential markers and therapeutic targets for cancer of the breast and prostate (US-A 2002/0127619), although changes in established cell lines or after transformation are frequently not significant. It is thus not possible in this way to achieve the aim of defining an early marker for tumor promotion.

Celis and coworkers (J. E. Celis, M. Ostergaard, B. Basse, A. Celis, J. B. Lauridsen, G. P. Ratz, I. Andersen, B. Hein. H. Wolf, T. F. Orntoft, H. H. Rasmussen, Cancer Research (1996) 56, 47824790) were able to show by 2D gel electrophoresis that adipocyte FABP (A-FABP) declines drastically in advanced stages of squamous cell carcinomas of the bladder. Advanced stages of bladder carcinogenesis were statistically correlated with the presence or absence of A-FABP. The authors conclude that A-FABP is an important component in the development of squamous cell carcinoma and therefore has prognostic value.

Fatty acid binding proteins were also notable in an analysis of peroxisome proliferator-induced proteomic changes where the coupling of these changes to a receptor mechanism was shown by comparative investigations in PPAR-α (peroxisome proliferator-activated receptor of type alpha)-knockout mice (Macdonald N., Chevalier S., Tonge R., Davison M. Rowlinson R., Young J., Rayner S. Roberts R., Arch. Toxicol (2001) 75, 415-424). An increase in the protein was also reported in this paper.

It is an object of the present invention firstly to provide a practicable method for testing substances or substance mixtures which allows carcinogenic and, in particular, tumor-promoting properties to be recognized.

This object is achieved by the present invention through determination of whether exposure of an organism or a part thereof to the substance to be tested or to the substance mixture to be tested alters the expression of at least one L-FABP. This is based on the finding with the aid of test substances having an effect unambiguously defined for the endpoint, such as tumor promoters, and their characterization in the long-term test, that L-FABP is a relevant marker which is present in sufficiently high concentration in healthy cells and is changed on treatment with substances. On the basis of the behavior of L-FABP found on exposure to tumor-promoting substances, the method of the invention has the advantage in particular of having a sufficiently high statistical significance, thus allowing such effects to be detected above the background noise. “Background noise” is defined in this connection as shifts in neighboring intensities of other proteins to whose change no causal connection can be assigned.

The present invention relates to a method for testing substances or substance mixtures, where
a) an organism or a part thereof is exposed to the substance or to the substance mixture; and
b) the expression of at least one liver fatty acid binding protein (L-FABP) is determined in at least one sample derived from the organism or the part.

In step a) of the method, an organism or a part thereof is exposed to the substance to be tested or to the substance mixture to be tested. The aim is to detect and, in particular, to quantify in step b) of the method the changes in L-FABP expression which are associated with the exposure.

It is advantageous according to the invention to carry out the expression analysis on at least two occasions. Repetition of the expression analysis at different times makes it possible to determine the relative change in L-FABP expression as a function of time. Such a time-dependent determination permits a significant change over time to be detected as a trend, with the aid of suitable statistical methods, thus allowing transient phenomena, for example an initial contrary change in L-FABP expression to be recognized and evaluated appropriately.

Accordingly, in an advantageous embodiment, the method of the invention comprises
a) exposing an organism or a part thereof to the substance or to the substance mixture; and
b) determining the expression of at least one liver fatty acid binding protein (L-FABP) in at least a first and at least a second sample derived from the organism or the part,
   where the first sample has been taken from the organism or the part thereof after a first exposure time and the second sample has been taken from the organism or the part thereof after a second exposure time, and the first exposure time is different from the second exposure time.

This embodiment of the method of the invention is expedient in particular when samples can be taken during the exposure time to the substance or to the substance mixture, i.e. the taking of samples does not terminate the exposure to the substance or to the substance mixture. This embodiment is thus particularly suitable for in vitro systems.

In a further advantageous embodiment, the method of the invention comprises
a1) exposing a first organism or a part thereof to the substance or to the substance mixture;
b1) determining the expression of at least one liver fatty acid binding protein (L-FABP) in at least one sample derived from the first organism or the part;
a2) exposing a second organism or a part thereof to the substance or to the substance mixture; and
b2) determining the expression of the liver fatty acid binding protein (L-FABP) in at least one sample derived from the second organism or the part,
   where the exposure time of the first organism or the part thereof is different from the exposure time of the second organism or the part thereof.

This embodiment of the method of the invention is particularly expedient when the exposure to the substance or to the substance mixture is terminated by the taking of samples. This applies in particular to in vivo systems, i.e. for example animal experiments in which the animal is sacrificed after a particular exposure time to the substance or to the substance mixture, and the animal or a part thereof intended for determining the expression is put into a condition which allows no further change in the expression of the liver fatty acid binding protein.

### a) The Exposure to the Test Substance or to the Test Substance Mixture

Exposure in vivo is preferred. If an organism is used for this purpose, it is preferably an animal organism, in particular a vertebrate, preferably a mammal, especially rodents, for example rats or mice. In a particular embodiment, a rat or mouse strain which is utilized in toxicology is used, for example Wistar rats or, preferably, Fischer 344 rats. The latter comprise an inbred strain which is expected to have low variability of the cellular protein pattern from animal to animal. The substance to be tested or the substance mixture to be tested is administered to the organism normally in a targeted manner, especially orally, for example with the feed or by gavage, or by injection, for example introperitoneally, intravenously, subcutaneously or intradermally.

The exposure time may in fact vary. However, firstly a minimum exposure time is important for the method of the invention, so that the effects caused by the exposure can be determined. Secondly, a relatively short exposure time is expedient so that the method can be carried out quickly. Thus, the time may range from a few hours to several days or even several weeks. However, preference is given according to the invention firstly to a minimum exposure time in the region of more than 24 hours, in particular of at least 36 hours and advantageously of at least 48 hours, and secondly to a relatively short exposure time of up to 10, 9, 8, 7, 6, 5 or 4 days and in particular of up to 72, 68, 64, 60, 56, 52, or 48 hours.

The combination, preferred according to the invention, of in vivo exposure and relatively short exposure times is associated in particular with the following advantages:
the possibility of being able to employ relatively small amounts of substance;
a short time for carrying out the method;
the use of a relatively small number of animals.

The method of the invention normally comprises choosing in a plurality of approaches different exposure times in order in this way to be able to recognize an exposure time-dependent change in L-FABP expression. An analogous statement applies to the dosage of the substance to be tested or of the substance mixture to be tested.

If a part of an organism is used, possible examples thereof are organs, tissue preparations or isolates thereof, in particular cell-containing fractions. These can be prepared in an expedient manner for ex vivo and in vitro assays. The exposure to the substance or to the substance mixture then corresponds to an incubation.

In a particular embodiment of the present invention there is use of liver or liver constituents, for example liver extracts or liver cells and liver cell cultures.

The exposure or the incubation is followed by certain parts of the treated organism, or the incubation mixture or parts of the incubation mixture being provided as sample, if necessary with suitable working up, for analytical protein determination.

### b) Analytical Protein Determination

The expression analysis of the invention comprises determination of protein expression and thus information about the amount of protein and protein composition present in the cell at the time of testing. This is important according to the invention. An mRNA analysis would not provide this information directly because there is no strict correlation between the amount of mRNA and the relevant amount of protein owing to translation regulation, mRNA stability, protein stability and protein degradation.

The term “liver fatty acid binding protein” (L-FABP for short) refers to proteins which are involved in the transport of fatty acids and other hydrophobic ligands. Appropriate for their designation, these proteins occur in the liver of vertebrates.

Because of differences in phylogenetic development, there is a certain species-dependent heterogeneity within this group of proteins. The determination will depend on the organism and will be directed at the particular L-FABP to be expected in the relevant organism. The determination is directed in particular at L-FABPs from the rat, in particular from Rattus norvegicus.

In addition to species-dependent variations, also found for each species are usually polymorphic variants which, owing to allelic variation, have different amino acid sequences. Moreover the group of L-FABPs also includes proteins of identical sequence but having different post-translational modifications such as particular glycosylation patterns.

In a particular embodiment of the present invention, the expression analysis is directed at an L-FABP having the amino acid sequence of SEQ ID NO: 1.

Further useful directions for the L-FABP determination of the invention can be found by the skilled worker or from the amino acid and nucleic acid sequences indicated in the aforementioned publications. In addition, there are numerous entries in relevant gene databases on L-FABP-encoding nucleic acid sequences, on the basis of which the skilled worker is able to provide suitable means for detecting the corresponding proteins.

The analysis of the invention is substantially divided into three steps of the method:

b1) expedient provision of the expression product to be determined;
b2) quantification of the expression product; and if appropriate
b3) evaluation.

Steps b1), b2) and b3) of the method are advantageously carried out in the stated sequence. If further investigations, for example determinations of further proteins, are carried out together with the L-FABP determination, these investigations can be carried out in separate methods or, in a preferred embodiment of the present invention, at least partly in parallel in an appropriately designed method, with in particular at least steps b1) and b2) of the method being carried out in parallel.

b1) Provision of The Expression Product (Sample) to be Determined

It is possible in principle to analyze any samples of the organism or of a part thereof. Body samples such as organs and tissues, native, frozen, fixed, with or without dissection, and especially cell-containing fractions thereof, and the incubation mixtures described above or parts thereof can advantageously be used for the L-FABP determination of the invention. Accordingly, this part of the method of the invention is an in vitro method.

In a preferred embodiment of the present invention, liver and liver constituents or isolates, especially cell-containing fractions thereof, are used as sample.

With a view to the expression analysis to be carried out according to the invention, if required the cellular constituents, and in particular the expression products to be determined, which are present in the sample undergo a preparative working up, thus providing them in an expedient form in relation to the method of the invention. Such a working up ordinarily corresponds to conventional practice and is based in particular on the requirements of analytical protein expression determination and especially of proteomic analysis.

The requirements for suitable sample preparation are usually strict. Artifactual alterations in the protein composition, for example through proteolysis or other modifications (e.g. oxidations), should be avoided.

If the sample is of tissue, this is usually initially homogenized. This is ordinarily followed by cell disruption. For this purpose, the sample can be exposed, for example, to shear forces or put into a hypotonic environment in which the cells are then ruptured by osmolysis. The latter can be brought about with conventional lysis buffers which should also usually comprise suitable protease inhibitors. The resulting lysate can then be provided for the actual analytical protein determination or initially stored at low temperature, for example at −80° C.

In order to be able to compare the analysis of a plurality of lysates with one another, the total protein content of each lysate can be determined by conventional methods. Usually suitable for quantitative determination are color assays such as the biuret assay, the Lowry assay, the bicinchoninic acid assay, the Bradford assay, and further spectroscopic methods or else determination of proteins by radiolabeling.

Appropriate aliquots of the lysates can be chosen on the basis of the total protein content, so that approximately equal amounts of total protein are supplied for the subsequent analysis.

b2) Quantification of the Expression Product

It is possible in principle to employ all methods known to be suitable for quantifying proteins from the areas of protein analysis and, in particular, proteomic analysis. Thus, for example, immunological techniques and certain spectroscopic methods, if necessary combined with chromatographic or electrophoretic separation methods, may be mentioned. In order to ensure specific detection of the expressed proteins, it is advantageous to use immunological methods. Usually required for this are antibodies which recognize the protein to be determined with maximal specificity.

Suitable L-FABP-recognizing antibodies have been disclosed and can in some cases also be obtained commercially. For example, antibodies both against human L-FABP and against rat L-FABP are available from HyCult Biotechnology b.v. These include both monoclonal and polyclonal antibodies, some of which are cross-reactive with L-FABP from other species. Thus, for example, a monoclonal antibody against human L-FABP (clone K5A6, catalog No. HM 2051), which is also available in biotinylated form (catalog No. HM 2052), a polyclonal antibody against human L-FABP (catalog No. HP 9021) and a polyclonal antibody against rat L-FABP (catalog No. HP 8010), which is cross-reactive with human, porcine and murine L-FABP, may be mentioned. Novocastra Laboratories Ltd. supply a monoclonal antibody against human L-FABP, which also reacts with the renal and intestinal fatty acid binding proteins.

The skilled worker is moreover able, starting from the L-FABP amino acid sequence, to produce suitable antibodies directed against the protein. It is possible for this purpose to use the complete protein or derivatives, e.g. fragments thereof (polypeptides), as immunogen and to produce in a manner known per se polyclonal and monoclonal anti-bodies and, based thereon, also humanized antibodies by recombinant techniques, and fragments thereof.

For example, it is possible to produce suitable antibodies by immunizing a host with at least one L-FABP of the invention or a derivative thereof, and isolating the host's antibody-containing serum produced as a response to the immunization.

If the L-FABP to be used has only low or no immunogenicity, it is possible to increase the immunogenicity by coupling it to a carrier, preferably a carrier protein such as KLH. A number of possibilities for coupling for this purpose are available to the skilled worker. It is possible and expedient for example to react with glutaraldehyde, for example by incubating the protein or a protein mixture with the carrier protein or a mixture of various carrier proteins in water or an aqueous solvent. The reaction ordinarily gives a desired result within a few hours. Optimization of the reaction parameters is within the capability of the skilled worker.

In addition to the antigen, immunization cocktails ordinarily comprise further auxiliaries, in particular adjuvants normally employed for immunization, e.g. Freund's adjuvant.

Rodents or else rabbits are particularly suitable as host. These or other suitable hosts receive the immunization cocktails by injection, preferably subcutaneously. The antibody titers can be determined by an immunoassay, for example competitive with a sheep antiserum directed against host IgG and labeled oligomer. It is thus possible to decide toward the end of the immunization whether a particular host is suitable for obtaining antibodies. If, for example, four immunizations are carried out, the antibody titer can be determined after the third immunization, and then antibodies can be obtained from animals having an adequate antibody titer.

To obtain the antibodies produced, it is preferred to take blood from the hosts over several weeks or months. Finally, the host can be exsanguinated. Serum comprising the desired antibodies can be obtained in a manner known per se from the blood obtained. The complete serum obtain in this way can if necessary be further purified in a skilled manner in order to concentrate the antibody fraction present therein and, in particular, the L-FABP-recognizing antibodies.

In a particular embodiment of this method there is selection of at least one antibody in the serum which specifically recognizes the L-FABP used as immunogen, a derivative thereof or at least one L-FABP present in the composition used as immunogen or a derivative thereof. Specificity means in this connection a higher binding affinity of the antibody for the immunogen than for other, especially related, proteins, in particular further FABPs as mentioned at the outset. Monoclonal L-FABP-specific antibodies can also be obtained in this way. However, for this purpose it is preferred to take spleen tissue from the host and, starting from the spleen lymphocytes obtained in this way, to establish in the usual manner hybridomas which produce the monoclonal antibodies.

The antibodies obtainable according to the invention include in particular antisera which can be obtained by the above methods. These may be complete sera, i.e. blood obtained from the host after removal of the cellular and coagulable constituents, or fractions of this serum in which in particular the immunoglobulin fraction and preferably the L-FABP-recognizing immunoglobulin fraction is enriched. Fractions of this type can be obtained by the methods described above in connection with antibody purification.

Polyclonal antisera comprise antibodies differing in specificity, ordinarily different classes and subclasses, and normally all L-chain isotypes are represented, and multiple protein epitopes are recognized.

The antibodies which can be obtained also include monoclonal antibodies, especially chimeric and humanized antibodies, and L-FABP-binding fragments thereof.

These antibodies can then be used in particular in quantitative immunoassays and immunoblotting techniques e.g. Western blotting. Both direct and indirect assays are suitable. Particular mention should be made of competitive immunoassays, i.e. the protein or polypeptide to be detected competes as antigen with labeled antigen for antibody binding. Sandwich immunoassays are preferred, i.e. the binding of specific antibodies to the antigen is detected using a second, usually labeled, antibody. These assays can be designed to be either homogeneous, i.e. without a separation into solid and liquid phase, or heterogeneous, i.e. bound labels are separated from unbound ones, for example by solid phase-bound antibodies. The various heterogeneous and homogeneous immunoassay formats can be assigned to particular classes depending on the labeling and method of measurement, for example RIAs (radio immunoassays), ELISA (enzyme linked immunosorbent assay), FIA (fluorescence immunoassay), LIA (luminescence immunoassay), TRFIA (time-resolved FIA), IMAC (immunoactivation assay), EMIT (enzyme multiplied immune test), TIA (turbidometric immunoassay).

It is moreover possible to obtain immunological assays for determining L-FABPs commercially. For example, HyCult Biotechnology b.v. supplies an appropriate ELISA assay kit which operates on the sandwich principle. Briefly, samples and standards are incubated in microtiter plates coated with L-FABP-recognizing antibodies. During the incubation, L-FABP is trapped by the antibody bound to the solid phase. Non-binding material present in the sample is removed by washing. Subsequently, a second, biotinylated antibody directed against L-FABP (tracer) is added. The tracer antibody binds to trapped L-FABP where present. Excess tracer is removed by washing. Subsequently, a streptavidin-peroxidase conjugate is added and reacts specifically with the biotinylated tracer antibody which is bound to L-FABP. Excess streptavidin-peroxidase conjugate is removed by washing. A substrate is then added, especially tetramethyl-benzidine (TMB). The color development is proportional to the amount of L-FABP present in the sample. The enzymatic reaction is stopped by adding citric acid, and the extinction at 450 nm is measured with a spectrophotometer. A standard curve is obtained by plotting the extinctions against the corresponding concentrations of the known standard. The L-FABP concentrations in the samples with unknown concentrations which are run in parallel to the standards can be read off the standard curve. Besides immunological methods it is also possible to use non-immunological, usually spectroscopic methods for quantitative determination of L-FABP.

However, since most spectroscopic methods on their own do not ensure specific detection of L-FABP, it is usually necessary to fractionate the total protein present in the lysate by relevant separation methods in such a way that specific detection of the L-FABP is possible by means of spectroscopic methods.

Chromatographic and electrophoretic methods are suitable for the fractionation. Suitable chromatographic methods include for example affinity chromatography. Electrophoretic methods include for example gel electrophoresis or capillary electrophoresis, both under denaturing and under native conditions, for example polyacrylamide gel electrophoreses, isoelectric focussing and the like.

In a particular embodiment of the method of the invention, the proteins are separated by two-dimensional gel electrophoresis. This is particularly suitable for proteomic analysis because it provides high resolution and can be carried out relatively quickly.

The first step carried out in two-dimensional gel electrophoresis is an isoelectric focussing (1st dimension), and the second step is an SDS polyacrylamide gel electrophoresis (2nd dimension). In some circumstances, sprayed pH gradients or prefractionations are used in order to separate common and rare proteins as far as possible from one another.

Proteins must be labeled for quantification in the gel matrix. Stainings with Coomassie blue and the more sensitive silver staining are usual. Detections of radiolabeled proteins and immunological labels are even more sensitive, concerning which reference may be made to the above statements concerning immunological methods.

Depending on the label, various detection systems are available to the skilled worker to quantify the stained proteins. In the case of two-dimensional gels, this usually takes place by densitometry, for example with a laser densitometer or a scanner. It is possible in this way to quantify the amount of L-FABP present in the sample, both in absolute terms and by comparison with further proteins present in the sample.

If necessary, the spot(s) corresponding to L-FABP can be identified. For example, the intact protein corresponding to one spot can be transferred from the gel matrix to a chemically inert membrane and there subjected to further protein chemical analysis. Alternatively, the protein in the gel matrix can be broken down into smaller fragments, for example enzymatically, and eluted, and the fragments can then be analyzed. The intact protein can be analyzed for example by carrying out an amino acid sequence analysis, or by means of mass spectrometry, in particular IR-MALDI mass spectrometry, the molecular weight of the protein can be determined and used to identify it. If the protein is first broken down into smaller fragments, this can be brought about by means of conventional enzymes, for example trypsin, LysC endoprotease and AspN endoprotease. Elution of the resulting peptide fragments from the gel matrix is usually possible with organic solvents and acids. Mass spectrometry is likewise suitable for analyzing the peptides, for example MALDI mass spectrometry or ESI (nanospray) mass spectrometry, if appropriate combined with a preceding HPLC separation of the peptides.

Of the mass spectrometric methods, further mention should be made of that called the SELDI method. This entails the protein mixtures to be investigated initially being trapped on suitable surfaces, e.g. solid support surfaces with affinity for proteins, if necessary unwanted substances being removed from the surfaces, for example by washing with suitable liquids, and subsequently determination taking place by MALDI-TOF (matrix assisted laser desorption/ionization time-of flight) mass spectrometry.

b3) Evaluation

It is possible with the measurement methods described above to assign to each investigated sample a particular value which characterizes the expression of L-FABP and indicates in particular the amount of L-FABP in the sample, either absolutely or by comparison with a standard, either internal or else externally added.

It is particularly important according to the invention to establish whether L-FABP expression is changed through exposure to the test substance or to the test substance mixture, or not. This requires comparison of the expression determined for a first dosage and/or a first exposure time with the expression of L-FABP in a sample from a corresponding organism or a part thereof which is not treated with the test substance or the test substance mixture, and/or has been exposed to the test substance or the test substance mixture in a second dosage different from the first, or for a time different from the first.

It is possible in principle to perform such a comparison by carrying out the L-FABP determination of the invention before and after exposure to the substance or to the substance mixture or after various exposure times and/or dosages, and comparing the amounts of L-FABP with one another. In some circumstances, it is also possible with an established test system to have recourse to comparative values deposited for example in a database, without the need to carry out the method or determination experimentally per se.

For validation of a particular test system it is expedient to establish a particular value (limiting value) above which by definition there is a significant change in expression.

Such a limiting value may depend on the nature of the investigated sample and also on the obtaining thereof. Thus, it is expedient to carry out a particular model system for implementing the method of the invention initially several times without previous exposure to test substances or test substance mixtures, and to find an appropriate average for L-FABP expression. It is then possible, with the aid of substances which are known to have the property to be determined using the method of the invention, and of the L-FABP values found for these substances using the method of the invention, to set expedient limits for assessing test substances or test substance mixtures.

The method of the invention is particularly aimed at assessing a toxic, especially carcinogenic and in particular tumor-promoting property of a tested substance. The properties to be assessed in particular include those which are receptor-mediated, enzyme-inducing, cytotoxic/mitogenic, oxidative stress-promoting and/or mitochondrially toxic.

In this connection, the method of the invention is advantageous because exposure to the test substance or to the test substance mixture, especially under the preferred conditions described above (in vivo exposure; relatively short exposure times) leads to a significant decrease in L-FABP expression when the test substance or the test substance mixture is toxic, namely carcinogenic and in particular tumor-promoting, and such a significant decrease can be detected with conventional, straightforward methods, e.g. immunological methods.

The present invention therefore relates further to the use of the method of the invention for the aforementioned purposes. This is connected in particular with the analytical finding of whether exposure to a substance or to a substance mixture leads to a change and, in particular, to a decrease in L-FABP expression. If this is so, the substance or the substance mixture has toxic, namely carcinogenic and in particular tumor-promoting properties.

The present invention also relates to analytical kits for carrying out the method of the invention. These normally comprise
i) at least one means for determining L-FABP expression, in particular specific antibodies; and if appropriate
ii) further usual means for carrying out the method of the invention.

Further particular embodiments of kits of the invention are evident from the statements about the method itself.

### DESCRIPTION OF THE FIGURES

The drawings show

FIG. 1 a typical appearance of a 2D gel of rat liver proteins (100 μg total protein, pH=4-7, 12.5% acrylamide, proteins stained with silver);

FIG. 2 contrast profiles of two proteins, IDNR=1168 and IDNR=1624, over 16 groups of male rats treated with phenobarbital;

FIG. 3 the (A) linear and (B) logarithmic scatter plot of the amounts of protein in two different rat livers from a time/dose group (2919 spots are “matched”, the correlation coefficient is 0.965);

FIG. 4 the intersection frequencies of the three assays PHEN_M, ETHI_F and HEXA_F separated into (A) RUN1 and (B) RUN2, the test criterion being the Hochberg-Benjamini adjusted 1% level;

FIG. 5 the plot of the average logarithmic spot intensities of the protein PHEN_M ID=3368, ETHI_F_ID=4302, HEXA_F_ID=4425 over 16 treatment contrasts, the error bars indicating the average standard error;

FIG. 6 a synthetic supermaster gel with the relevant spot_IDs ETHI_F_ID 4302 and ETHI_F_ID 4316, both of which represent L-FABP;

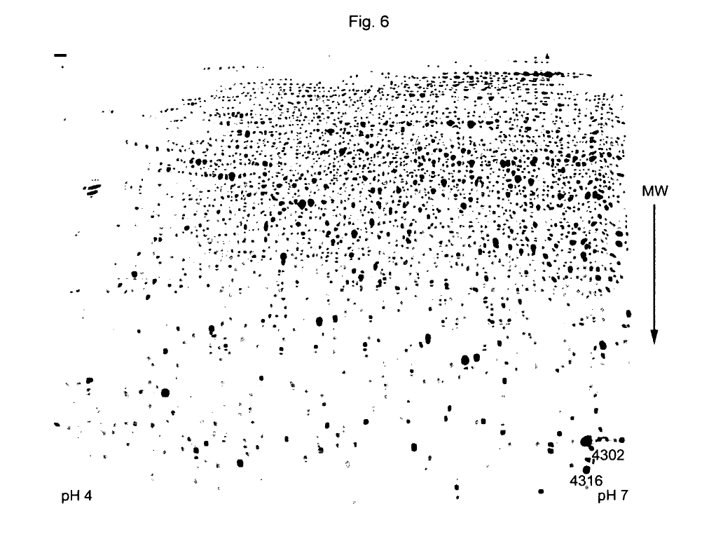

FIG. 7 a Western blot in which L-FABP is visualized with an anti-L-FABP rat antibody and a chemiluminescence-labeled anti-rat antibody (lane 1: rainbow marker 1:1 with SDS sample buffer; lane 2, 3: control after 3 days; lane 4, 5: rats treated with phenobarbital (high dosage) for 10 days; lane 6, 7: control after 10 days; lane 8, 9: female rats treated with ethinylestradiol (high dosage) for 10 days; lane 10: SDS sample buffer);

FIG. 8 a Western blot in which L-FABP is visualized with an anti-L-FABP rat antibody and a chemiluminescence-labeled anti-rat antibody (lane 1: rainbow marker 1:1 with SDS sample buffer; lane 2, 3, 6, 7: control after 10 days; lane 4, 5, 8, 9: female rats treated with alpha-hexachlorocyclohexane (high dosage) for 10 days; lane 10: SDS sample buffer);

FIG. 9 a Western blot in which L-FABP is visualized with an anti-L-FABP rat antibody and a chemiluminescence-labeled anti-rat antibody (lane 1: rainbow marker 1:1 with SDS sample buffer; lane 2, 4, 6, 8: control after 10 days; lane 3, 5: female rats treated with tetrachloromethane (high dosage) for 10 days; lane 7, 9; female rats treated with furan (high dosage) for 10 days; lane 10: SDS sample buffer);

FIG. 10 a Western blot in which L-FABP is visualized with an anti-L-FABP rat antibody and a chemiluminescence-labeled anti-rat antibody (lane 1: SDS sample buffer; lane 2, 4, 6, 8: control after 10 days; lane 3, 5: female rats treated with 2,6-dinitrotoluene (high dosage) for 10 days; lane 7, 9,: female rats treated with 2,4-dinitrotoluene (high dosage) for 10 days lane 10: rainbow marker 1:1 with SDS sample buffer);

FIG. 11 a Western blot in which L-FABP is visualized with an anti-L-FABP rat antibody and a chemiluminescence-labeled anti-rat antibody (lane 1: rainbow marker 1:1 with SDS sample buffer; lane 2, 4, 6, 8: control after 10 days; lane 3, 5:

female rats treated with 2,6-diaminotoluene (high dosage) for 10 days; lane 7, 9: female rats treated with 2,4-diaminotoluene (high dosage) for 10 days lane 10: rainbow marker 1:1 with SDS sample buffer);

FIG. 12 a Western blot in which L-FABP is visualized with an anti-L-FABP rat antibody and a chemiluminescence-labeled anti-rat antibody (lane 1: rainbow marker 1:1 with SDS sample buffer; lane 2, 4, 6, 8: control after 10 days; lane 3, 5: female rats treated with WY 14,643 (high dosage) for 10 days; lane 7, 9: female rats treated with cyproterone acetate (high dosage) for 10 days lane 10: SDS sample buffer);

FIG. 13 a Western blot in which L-FABP is visualized with an anti-L-FABP rat antibody and a chemiluminescence-labeled anti-rat antibody (lane 1, 7, 8, 9, 10: SDS sample buffer; lane 2: rainbow marker 1:1 with SDS sample buffer; lane 3, 5: control after 10 days; lane 4, 6: female rats treated with nafenopin (high dosage) for 10 days.

### EXAMPLES

### 1. Animal Exposure to the Test Substances

The animal experiments are carried out with Fischer 344 rats. This rat strain is an inbred strain expected to have less variability of effects from animal to animal compared with other strains used in toxicology, such as Wistar. 5 animals (female; in the phenobarbital group additionally the same number of male animals) are employed per time point in each experiment for each dose level. Exposure takes place for 4 or 17 hours and 3 or 10 days. A control group of 5 animals is included for each time period and receives either the vehicle for the test substance solution (corn oil or dd water) or substance-free feed. A low, intermediate and a high dose are used for each time point. Thus, 80 animals are employed for each experiment. The substance is administered by gavage for the two short time periods with a single administration at the start of the time window. For the 3- and 10-day exposures, the animals receive the test substance in the feed. Exposure to the highly bioaccumulating alpha-hexachlorocyclohexane is an exception. In this case, the animals exposed for 3 and 10 days receive an initial dose by gavage and then a maintenance dose of 10% of the initial dose in the feed. At the end of the exposure time, the animals are anesthetized with gaseous carbon dioxide and exsanguinated by decapitation. The livers are removed as quickly as possible, divided into segments, shock-frozen in liquid nitrogen and stored deep-frozen until analyzed. The kidneys are preserved as control organs without further division in the same way, but not investigated further in this example.

### 2. Rat Liver Sample Disruption

For the cell disruption, a deep-frozen liver segment is firstly cooled further in liquid nitrogen. It is then crushed with the aid of a metal pestle. Aliquots each of about 47-52 mg are distributed into 2 ml Eppendorf tubes and stored at −80° C. This avoids partial thawing of the liver fragments when forming aliquots for each sample disruption.

For the actual sample disruption, two aliquots of the same sample are taken and mixed with lysis buffer (42.04 g of urea, 15.22 g of thiourea, 4.0 g of CHAPS, 1.0 g of DTT, 2 ml of Ampholine pH 3.5-10, 48 mg of Pefabloc SC, 48 mg of EDTA, 50 μg of leupeptin, 70 μg of pepstatin, 100 μg of aprotinin; with WFI to 100 ml). For both samples, 50 mg of liver cells are mixed with 1000 μl of buffer. The same amount of glass beads (glass beads No. 2; from Buddeberg, order number 22.222.0002), which corresponds to the mass of the respective initial weight of rat liver plus lysis buffer, is added to this mixture without delay. The samples are then homogenized with an oscillating mill (30′, full power) in a cold room at +4° C. The sample cups of the oscillating mill are cooled to −20° C. before the samples are inserted.

The homogenizing in the oscillating mill is followed by an incubation period of 60 minutes for the proteomic analytical determination and of 30 minutes for the immunological determination. Mixing is repeated occasionally during the incubation by inverting the Eppendorf tube.

After this time has elapsed, the samples are centrifuged at 22 000 rpm for 90 minutes at 20° C. in an HFA 22.2 rotor of a Heraeus Biofuge 28RS centrifuge. The two supernatants are then cautiously removed and combined in a 1.5 ml Eppendorf tube. The residues are discarded. The supernatant is then centrifuged again in an HFA 28.1 rotor at 28 000 rpm (45 000×g) and 20° C. for 60 minutes. The supernatant is then cautiously transferred into a new 1.5 ml Eppendorf tube and finally stored in 80 μl aliquots at −80° C.

### 3. Protein Determination

### 3.1. Lowry Method

Before the electrophoresis, a protein determination by the Lowry method is carried out on each individual sample so that the same amount of protein can later be loaded onto each 2D gel. The protein assay kit (Sigma protein assay kit, order number: P 5656) is used for protein determination on the rat liver extracts. This entails, before the actual determination of the protein content, all the proteins being precipitated by TCA precipitation. Possible interference with the measurement method by, for example, urea, DTT or CHAPS is avoided thereby, because they are removed with the supernatant in the precipitation.

1.5 ml Eppendorf tubes are prepared with appropriate inscriptions (samples, 5 standards and a blank). 20 μl are taken from the liver extracts, mixed with 980 μl of deionized water and homogenized on a vortexer. For the standard series, firstly the contents of a standard bottle (BSA) of the protein kit is dissolved with the required amount of deionized water. All further solutions are made up as described in the package leaflet for the Sigma protein assay kit. The BSA standard series is then prepared in analogy to Table 1:

TABLE 1
Mixture for the BSA standard series

| Protein standard solution | | Protein concentration |
|---|---|---|
| (μl) | Deionized water (μl) | (μg/ml) |
| 125 | 875 | 50 |
| 250 | 750 | 100 |
| 500 | 500 | 200 |
| 750 | 250 | 300 |
| 1000 | 0 | 400 |

The appropriate BSA stock solution is added to the water. The water is thoroughly mixed using a vortexer. 1000 μl of deionized water are pipetted into a 1.5 ml Eppendorf tube as blank.

100 μl of DOC (deoxycholate) are added to each of the different Eppendorf tubes (sample, standard and blank), homogenized and incubated at room temperature for 10 minutes. Then 100 μl of TCA (trichloroacetic acid) are added and thoroughly mixed. The sample vessels are centrifuged at 45 000×g for 10 minutes. The supernatants from the centrifugation are cautiously decanted off and discarded. The residues are each dissolved in 1 ml of Lowry reagent solution. These solutions are then transferred into macro cuvettes (macrocuvettes from Greiner, order No.: 61 41 01) already prepared for the spectroscopic measurement. The Eppendorf tubes are then rinsed with 1 ml of deionized water. The rinsing solution is added with stirring using a stirring bar to the Lowry solution in the cuvettes. The sample solutions are incubated at room temperature for 20 minutes. Then 500 μl of Folin & Ciocalteu's phenol reagent working solution are put in each cuvette. A stirring bar is used to mix thoroughly. After standing for a further 30 minutes, the samples are measured against the blank sample at 750 nm in a UVNIS spectrometer. The absorptions of the standard samples are plotted against the respective BSA concentration in a calibration plot. The protein concentration in the rat liver samples is found with the aid of this calibration plot.

### 3.2. Popov Method

The protein content of the individual extracts was determined by the Popov method (N. Popov. M. Schmitt, S. Schulzeck, H. Matthies, Acta biol. med. germ. 34, pp. 1441-1446 (1975)) for the immunological determination.

### 4. Proteomic Analysis

### 4.1. Isoelectric Focussing (IEF)-1st Dimension

### 4.1.1. Rehydratration

For rehydration of the IEF strips (Immobiline DryStrip pH 4-7, 24 cm, from Amersham Pharmacia, order number: 17-6002-46), fresh rehydration buffer (8 M (14.41 g) urea, 2 M (4.57 g) thiourea, 20 mM (92.52 mg) dithiothreitol (DTT), 1% (300 mg) CHAPS, 156 μl of IPG buffer pH 3-10 (from Amersham Pharmacia, order No.: 17-6000-87) ad 30 ml with WFI) is made up. A volume of the rat liver lysate corresponding to 100 μg is then removed, put into a 1.5 ml Eppendorf tube and diluted to a total volume of 600 μl by adding rehydration buffer. The solution is thoroughly mixed with a vortexer. The solution is then put into one of the elongate slots of the Immobiline DryStrip Reswelling Tray. The tray is leveled before use by means of the knurled-screws. The protective film is then taken off the IEF strip and the strip is placed with the gel side downward into the slot with the rehydration buffer/sample mixture. After all the gel strips have been inserted, the chamber is closed and sealed with adhesive tape for better sealing. The rehydration takes place at RT for 24 hours.

### 4.1.2. Isoeletric Focussing

After 24 hours, the chamber is opened, and the first gel strip is removed and dabbed on a filter paper (Whatman filter paper No. 3, order No.: 1003-917) moistened with WFI. This procedure takes place analogously for all the strips. The IEF strips are inserted with the gel side upward into the Immobiline DryStrip Aligner on the Pharmacia Multiphor chamber. Two electrode strips are moistened with WFI. The excess water is removed by dabbing on a paper wipe. An electrode strip is placed across all the gel strips both on the cathode side and on the anode side. The electrode strips are brought to the correct length before being applied. The electrodes are then put in place, covered with a layer of 80 ml of cover fluid (DryStrip Cover Fluid, from Amersham Pharmacia, order No.: 17-1335-01), the connections made and the chamber closed. The isoelectric focussing is carried out with the parameters listed in Table 2.

TABLE 2
Parameters for the isoelectric focussing in the Multiphor chamber

| Voltage | Current strength | Power | | Volt-hours |
|---|---|---|---|---|
| (V) | (mA) | (W) | Mode | (Vh) |
| 500 | 1 | 5 | gradient | 500 |
| 500 | 1 | 5 | gradient | 2500 |
| 3500 | 1 | 5 | gradient | 10 000 |
| 3500 | 1 | 5 | gradient | 45 000 |

The next morning, after about 25-30 kVh, the electrode strips are changed. For this purpose, the voltage part is put into pause mode, and the chamber is opened. The electrode bridges are removed and the old electrode strips are cautiously removed. Then new electrode strips moistened with WFI are inserted. The electrode bridges are replaced, the chamber is closed, and the voltage part is again set in RUN mode. After the run is complete, the voltage part is switched off, the chamber is opened and the electrode bridges, and the electrode strips, are cautiously removed. The gel strips are then dabbed on filter paper moistened with WFI in order to remove the adherent cover fluid. Subsequently, if the gel strips are not used directly for the second dimension they are stapled in a DIN-A4 plastic sleeve and stored at −80° C.

### 4.2. SDS Polyacrylamide Gel Electrophoresis (PAGE)-2nd Dimension

### 4.2.1. Preparation of the Ettan DALT-II Chamber

Firstly, the running chamber is charged with 7.5 I of WFI, and the control device of the chamber is switched on. The circulating pump is activated and the anode buffer concentrate (75 ml) is introduced. The SDS gels (Ettan DALT II Gel (12.5%): from Amersham Pharmacia, order No.: 17-6002-36, Ettan DALT II buffer kit, from Amersham Pharmacia, order No.: 17-6002-50) are inserted with 2 ml of gel buffer into the gel frames (gel side toward the glass plate) and the excess gel buffer is removed with a commercially available wallpaper roller. After the frame has been closed, residues of excess buffer are removed by inclining the latter. The channels at the left and right edge of the gels are then closed with agarose melted at 85° C. Subsequently, the frames are wetted at the lower end with WFI and inserted into the Ettan DALT chamber. The gels are then covered up to the mark with cathode buffer concentrate diluted 1:10.

### 4.2.2. Equilibration of the Gel Strips

The strips are placed with the gel side upward in the equilibration tray and, in the first step, 4 ml of DTT equilibration buffer (4 ml of equilibration stock buffer (6 M (36 g) urea, 30% (30 g) glycerol, 2% (2 g) SDS, 3.3 ml Tris-HCl buffer of pH 8.8 (1.5 M (18.2 g) Tris/HCl, 0.4% (0.4 g) SDS, pH 8.8 ad 100 ml with WFI), ad 100 ml with WFI)+20 μl of bromophenol blue solution (30 mg of bromophenol blue in 10 ml of Tris/HCl buffer pH 8.8)+200 μl DTT+1 ml of WFI)) are added to each. The tray is then agitated horizontally in a laboratory shaker for 15 minutes. The buffer is then cautiously decanted off. Horizontal shaking is then repeated with 4 ml of iodoacetamide equilibration buffer (4 ml of equilibration stock buffer+20 μl of bromophenol blue solution (cf. equilibration buffer)+260 mM (192 mg iodoacetamide)) for 15 minutes. The buffer is cautiously poured off. The gel strips which are now equilibrated are freed of excess equilibration buffer on a filter paper moistened with WFI.

### 4.2.3. Electrophoretic Run

The equilibrated gel strips are then cautiously inserted, with the support sheet side facing the glass plate, into the gap between the glass plate of the gel frame and the support sheet of the DALT gel and lowered into the buffer. The gel strips are positioned with the aid of the thin fluorescent ruler and gently pressed onto the DALT gel. Any air bubbles are also removed thereby. The chamber is then closed and the run is started with the parameters in Table 3.

TABLE 3

| | | Power per | Temp. | Time | |
|---|---|---|---|---|---|
| Stage | Pump | gel (W) | (° C.) | (Min.) | Remarks |
| 1 | Auto | 4 | 25 | 75 | constant |
| | | | | | power |
| 2 | Auto | 14 | 25 | 360 | constant |
| | | | | | power |

After the run is complete, i.e. the bromophenol blue front has reached the lower edge of the gel, the voltage is switched off and the chamber is opened. The gels are taken out of the gel frames and shaken with fixing solution (50% deionized water, 40% methanol and 10% glacial acetic acid) for at least two hours, but usually overnight.

### 4.3. Silver Staining of the Gels

The silver staining of the gels took place on the basis of the individual steps listed in Table 4.

### 4.3.1. Automatic Stainer

The protocol described in Table 4 was carried out in an automatic stainer in order to speed up the staining and increase reproducibility.

TABLE 4
Silver staining of proteins after 2D PAGE

| Step | Solution | Incubation time | |
|---|---|---|---|
| Fixing | 40% methanol | >1 | hour |
| | 10% acetic acid | | |
| | 50% deion. H₂O | | |
| Washing | 70% deion. H₂O | 20 | min |
| | 30% ethanol | | |
| Washing | 70% deion. H₂O | 20 | min |
| | 30% ethanol | | |
| Washing | 70% deion. H₂O | 20 | min |
| | 30% ethanol | | |
| Incubation | thiosulfate: 0.02% | 1 | min |
| Washing | deion. H₂O | 1 | min |
| Staining | AgNO₃; 0.2% | 20 | min |
| Washing | deion. H₂O | 20 | sec |
| Washing | deion. H₂O | 20 | sec |
| Development | Na₂CO₃: 3% | 3-5 | min |
| | formaldehyde: 0.05% | | |
| | thiosulfate: 0.0004% | | |
| Washing | deion. H₂O | 1 | min |
| Stopping | EDTA: 2% | 5 | min |
| Washing (5x) | deion. H₂O | 10 | min |
| Preservation | glycerol: 2% | >30 | min |

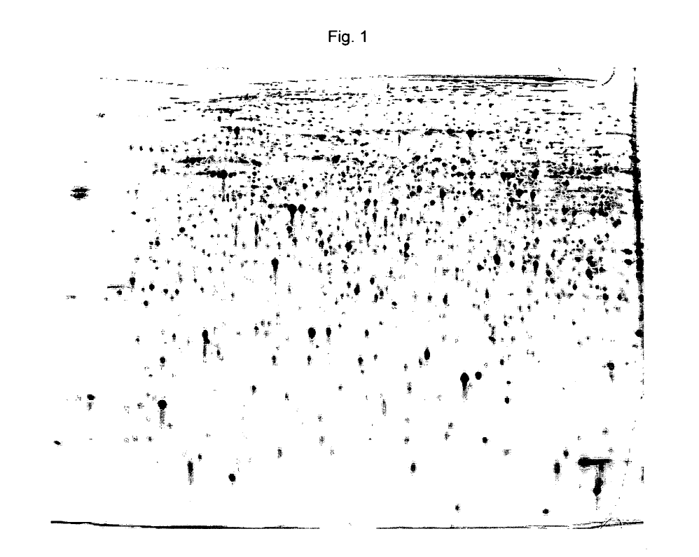

FIG. 1 shows a typical silver-stained 2D gel of rat livers in the analyzed pH range from 4 to 7.

### 4.4. Evaluation of the 2D Gels: Spot Detection, Quantification, Matching, Master Gels

After digitization of the gels with a 12 bit gray-scale scanner (Agfa Arcus II, 300 lines per inch) they are subjected to image analysis. The Melanie 3 software package from Genebio was employed for this. Firstly, all the spots were detected automatically. With an average of more than 3000 proteins per gel, elaborate manual re-editing is necessary in the regions of high protein concentration. The spots are quantified automatically after the detection.

The gels are subsequently subjected to matching. This entails generating a synthetic master gel which comprises all the proteins detected in the experiment and generating a common identification number (master ID) for each protein. In this connection too, all commercial software packages operate only incompletely: because of the distortions occurring in the gel, the matching must be checked manually and improved if necessary.

In the first experiment (phenobarbital, female animals), Melanie 3 produces a sub-master gel in each time-dose range. A master gel is then generated from all sub-master gels. However, it emerges from this that the number of mismatches is too high. The number of mismatches corresponds to the number of unique responders, by which is meant proteins which are found only in one treatment group. From the statistical viewpoint, such treatment contrasts are extremely unfavorable because they considerably underestimate the experimental error. For this reason, this experiment was initially not included in the evaluation.

In the evaluation of the next group (phenobarbital, male animals; alpha-hexachlorocyclohexane, female animals; ethinylestradiol, female animals), a different procedure is chosen: a master gel is produced for each substance from in each case two gels from the intermediate time/dose range. Then each individual gel of the group is matched on this master gel. Additionally appearing spots are added up.

To generate a “super master gel”, all master gels are matched on the master gel for ethinylestradiol. A final table matches the individual master IDs with the corresponding super master IDs.

### 4.5. Statistics

The aim of the statistical analysis of the data is a consistent assessment of the treatment contrasts. To do this it is firstly necessary to subject the underlying data design to detailed inspection. Each of the three assays PHEN_M, ETHI_F, HEXA_F (short for the phenobarbital/male, ethinylestradiol/female and alpha-hexachlorocyclohexane/female assays, respectively) are based on two 4*2 full-factorial experimental designs in the factors of DOSE (D) and TIME (T). An i*j-factorial design refers in this notation to an experimental design in which the first factor has been varied at i levels and the second factor has been varied at j levels. Alternatively, an i*j-factorial experimental design can be regarded as a two-dimensional data matrix with the first dimension at i levels and the second dimension at j levels (compare, for example, Table 5). Each of the two factors DOSE and TIME is varied at four D={D₁, D₂, D₃, D₄} or two levels T={T₁, T₂}, the time levels for all three assays consistently being “4 h”, “17 h”, “3 d” and “10 d”, while the dosage regimens differ between the three assays. The treatment regimen for the three assays is summarized in Table 5.

TABLE 5
summary of the treatment regimens of the three
assays PHEN_M, ETHI_F, HEXA_F

| | PHEN_M | ETHI_F | HEXA_F | Units |
|---|---|---|---|---|
| 4 h | 0 10 50 100 | 0 0.1 1 10 | 0 10 50 200 | mg/kg |
| 17 h | 0 10 50 100 | 0 0.1 1 10 | 0 10 50 200 | mg/kg |
| 3 d | 0 100 500 1000 | 0 1 10 100 | 0 10 50 200 | ppm* |
| 10 d | 0 100 500 1000 | 0 1 10 100 | 0 10 50 200 | ppm* |

*ppm: parts per million in the feed: conversion: 0.1* ppm = mg/kg of body weight

Both designs can be regarded as 4×2 high-dose/short-time and 4×2 low-dose/long-time designs, and it is statistically and biologically sensible to assess these two sub-designs—called RUN1 and RUN2 hereinafter—separately.

Overall, therefore, there are 16 treatment groups in each assay, and 5 independent repeats in each treatment group, i.e. each assay is represented by 80 gels. However, owing to missing values, there are fewer than 80 gels in all three assays. Each of these gels comprises k different proteins which were determined at i different times and j different dosages in the Ith repeat, i.e. the data consist of the indicated integral spot intensities Yₖᵢⱼₗ.

Exploratory preliminary inspection of the spot intensities Yₖᵢⱼₗ revealed that the average spot intensities of the control group with D=O are inhomogeneous, for which reason all 80 gels of an assay are, before further processing, centered on a common mean according to

Y′ₖᵢⱼₗ=Yₖᵢⱼₗ−Y_{•.ijl,}

in which Y_{•.ijl} refers to the average spot intensity of the 80 individual gels. This centering eliminates in a natural manner effects of over- and understaining between the gels without substantially influencing the effect structure.

The data preprocessed in this way are further examined in accordance with the underlying data structure using analysis of variance (ANOVA for ANalysis OF VAriance) methods in order thus to assess the treatment contrasts for significance protein-wise. For this purpose, the response Y′ₖᵢⱼₗ is broken down by analysis of variance according to

Y′ₖᵢⱼₗ=μₖ+αₖᵢ+βₖⱼ+γₖᵢⱼ+εₖᵢⱼₗ (1.0).

In this, μₖ is the global average of the kth protein over all treatments, αₖᵢ and βₖⱼ are the contribution of the ith dose level and γₖᵢⱼ is the synergistic contribution to the response Y′ₖᵢⱼₗ. The term εₖᵢⱼₗ is the error, which is assumed to have a normal distribution with constant variance σₖ², i.e. εₖᵢⱼₗ≈N(0, σₖ²).

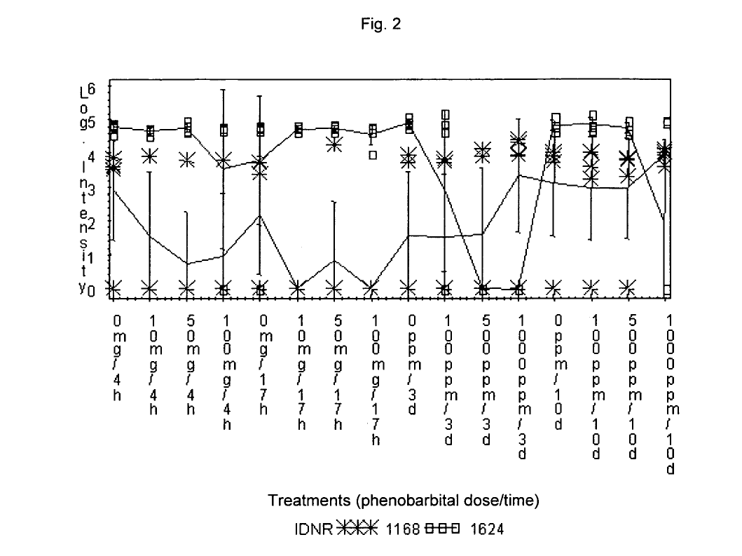

However, initial preliminary examinations of the data by analysis of variance show that simple ANOVA methods are not well suited for the available data. In order to illustrate these difficulties, the intensity contrasts of two proteins are plotted as representative in FIG. 2. The logarithmic integral spot intensity of the proteins IDNR=1168 and IDNR=1624 is plotted over all 16 treatment groups together with the standard Errors of Mean as error bars and measure of experimental variance.

A number of zero responders are evident in FIG. 2 and lead in the ANOVA to an extreme inflation of the error variance and thus conceal treatment effects. These zero responders are regarded as artefacts of the matching process, i.e. the protein spots in these cases were mismatched in individual gels—probably owing to distortion effects.

In order to make it possible to assess the data sensibly in the presence of extreme “outliers”, it is thus necessary to abandon the assumption of normal distribution and have recourse to the class of distribution-free or non-parametric methods. It is helpful for this that great advances have been made in recent years in the distribution-free analysis of factorial designs. In full-factorial experimental designs with k factors at nk levels, all combinatorial possible factor level combinations, i.e. (nₖ)^{k} combinations, are achieved. These designs allow polynomial effects to be identified up to the order (nₖ−1) and, in particular, allow interactions (synergisms) to be identified (see, for example, Brunner, E.; Puri, M. L. Nonparametric methods in design and analysis of experiments, Handbook of Statistics 13 (1996) 631-703). The core of the method consists of rank transformation of the original scale Y′ₖᵢⱼₗ protein-wise (k-wise) over the treatments and repeats, i.e.

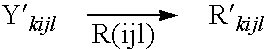

where R(ijl) indicates the rank formation over the classes i,j,l.

The non-parametric ANOVA problem can now be written analogously

R′ₖᵢⱼₗ=μₖ+αₖᵢ+βₖⱼ+γₖᵢⱼ+εₖᵢⱼₗ (2.0),

where the variances are now inhomogeneous over the classes k,i,j, i.e. εₖᵢⱼₗ≈N(0, σₖᵢⱼ²).

The free parameters of the mixed model (eq. 2.0) can be determined by maximum likelihood methods and allow the null hypothesis H0: (αₖᵢ=0, βₖⱼ=0, γₖᵢⱼ=0) to be tested against the alternative hypothesis H1: (αₖᵢ≠0 or βₖⱼ≠0 or γₖᵢⱼ≠0).

Depending on the particular number of protein spots, the described method leads to a very large number of individual tests, each of which tests has been assessed at 1% significance level a. In order to check the error of the 1st kind, α, of the overall assay, it is necessary to adjust these individual significance levels.

For this purpose the Hochberg-Benjamini false discovery rate method (Benjamini, Y. and Hochberg, Y, Controlling the False Discovery Rate: A Practical and Powerful Approach to Multiple Testing. Journal of the Royal Statistical Society B, 57 (1995) 289-300) is used, and an adjusted test level of 1% is used as basis for the three assays.

### 4.6. Protein Identification

The proteins of interest are each identified three times from independent Coomassie-stained preparative 2D gels. The livers of two female and one male rat were used for this purpose. The corresponding protein spots are cut out and cleaved enzymatically with trypsin in the gel. The resulting peptides are then analyzed by Nano-LC-MS/MS.

### 5. Immunological Determination by Means of Western Blotting

In order to improve pipettability, the rat liver extracts were initially mixed 1:10 with SDS sample buffer, homogenized with a vortexer and boiled for 10 minutes at 95° C. This was followed by renewed dilution with SDS sample buffer to a final protein concentration of 0.5 μg/μl. 4-12% Novex bis/tris gels were run with in each case 20 μl (sample, control, marker or buffer) per lane (gel running conditions: MOPS buffer; I=100 mA, P=50 W, t about 90 min) and blotted (blotting conditions: 0.2 μm PVDF membrane, from Invitrogen; U=200 V, I=102 mA, t=75 min.)

Detection took place in analogy to the method of the BM Chemiluminescence Western blotting kit (mouse/rabbit) from Roche Diagnostics. The anti-FABP antibody (from abcam Ltd., order No.: ab7847-500, rat-liver from rabbit, polyclonal) was diluted 1:500, and the anti-rat rabbit antibody was diluted to 40 mU/ml.

Also employed, to amplify the signal, was the ECL plus Western blotting detection system from Amersham Biosciences. The chemiluminescence was measured using a photon-counting camera. The integration time was 2.5 or 5 minutes.

### 6. Results

### 6.1. Proteomic Analysis

Of the groups of female (12) and male rats (1) exposed to a total of twelve non-genotoxic, tumor-promoting substances, four groups are investigated by proteomic analysis, namely the female animals treated with ethinylestradiol, alpha-hexachlorocyclohexane and phenobarbital, and the male animals likewise treated with phenobarbital.

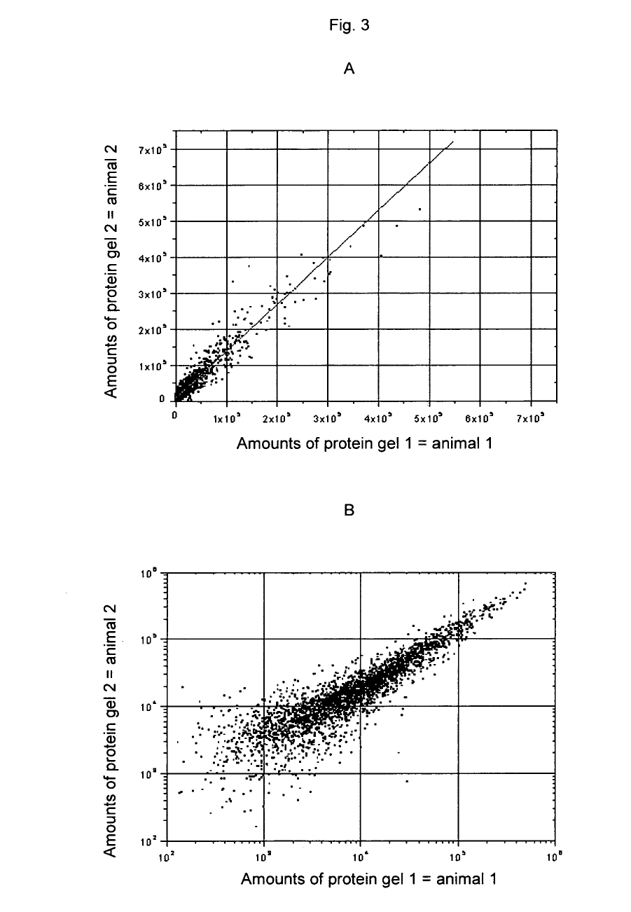

The reproducibility of the 2D electrophoresis is checked with the aid of a scatterplot. This entails the amounts of protein ascertained using Melanie 3 from two gels for each matched protein being plotted against one another. FIG. 3 shows such a scatterplot by way of example. With two gels each (corresponds to two different animals) from a time/dose group, the correlation of the integral amounts of protein is usually greater than 96%. The remaining difference represents the total of gel-to-gel variations and interindividual differences. This high reproducibility makes it possible to operate with only a single gel for each liver. Pooling of several livers from a time/dose group is dispensed with because important statistical information about the intersubject variation was lost thereby. Pooling would, however, have the advantage of a substantially smaller number of 2D gels.

Following the image analysis with spot detection, integration and matching, a synthetic master gel is generated for each substance group with Melanie 3. The matching method (phenobarbital, female animals) initially employed can be used only with provisos because the resulting error is too large. Every mismatch generates an additional spot in the gel. Instead of the approximately 3000 spots present on each single gel, the master gel shows 9317 spots.

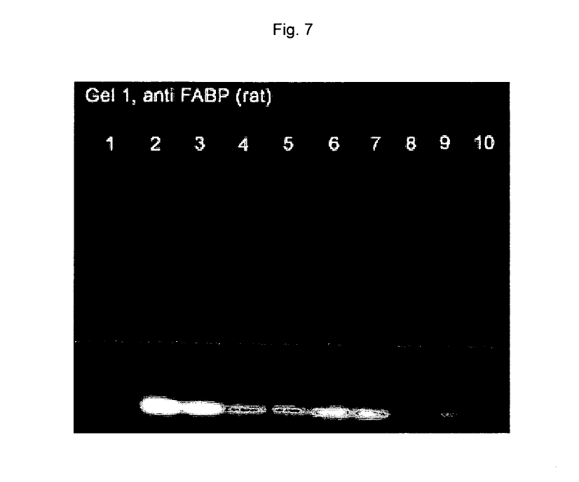

The simpler and less elaborate second matching variant results in master gels which show for the three remaining groups 3306 proteins with phenobarbital (male animals), and for the female animals 4277 protein spots with alpha-hexachlorocyclohexane and 4161 with ethinylestradiol (see Table 6). The synthetic ethinylestradiol master gel shown in FIG. 7 is used as basis for the super master gel.

Textiles are then generated from the three master gels and comprise the master protein ID in the first column, and the numerical integrals of the individual amounts of protein for each gel from the group in the remaining 80 columns. An additional table matches the individual master IDs with the corresponding super master IDs.

The three data sets were preprocessed using the method described in section 8, and the treatment contrasts were assessed for statistical significance. Table 6 gives a survey of the number of proteins on which the three assays are based, and the number of significant contrasts found in the statistical tests. In this connection, the data were utilized separately for the short time/high dose (RUN1) and long time/low dose (RUN2) treatment regimen.

It is directly evident from the comparison of RUN1 with RUN2 in Table 6 that treatment regimen 2—low dosage over longer periods—has a higher effect/noise ratio and thus, in biological terms, is also more effective. This is not entirely unexpected because under these conditions variable effects of the rise in level (caused inter alia by an initial high “bolus” dose) are replaced a wider time distribution of uptake, and thus peak effects progressing to an acutely toxic range are avoided.

TABLE 6
Total number and number of treatment contrasts assessed as significant
at the Hochberg-Benjamini adjusted 1% significance level

| | Number of proteins | N_{significant} | N_{significant} |
|---|---|---|---|
| Assay | Number | RUN1 | RUN2 |
| PHEN_M | 3306 | 9 | 407 |
| ETHI_F | 4161 | 92 | 112 |
| HEXA_F | 4277 | 22 | 38 |

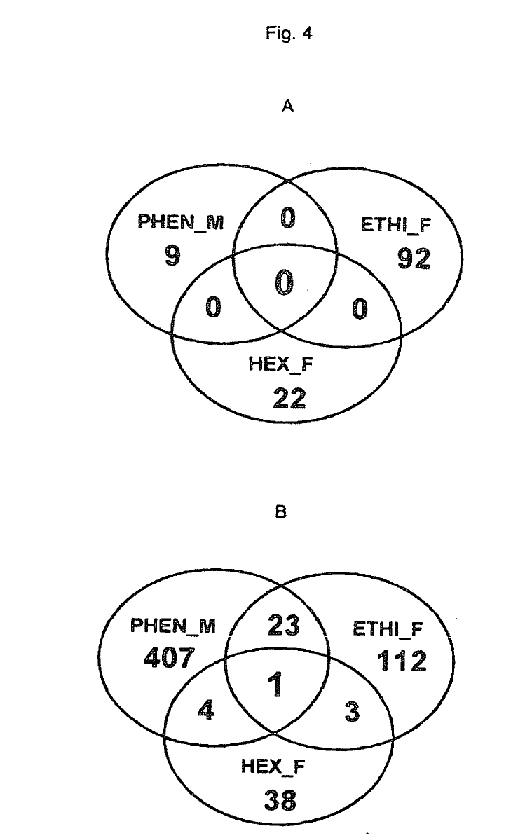

FIG. 4 is a diagrammatic illustration of the intersection frequencies of the three assays. This shows that all the intersections in RUN1 are empty, i.e. none of the proteins assessed as significant in an assay is found in the complementary assays and vice versa.

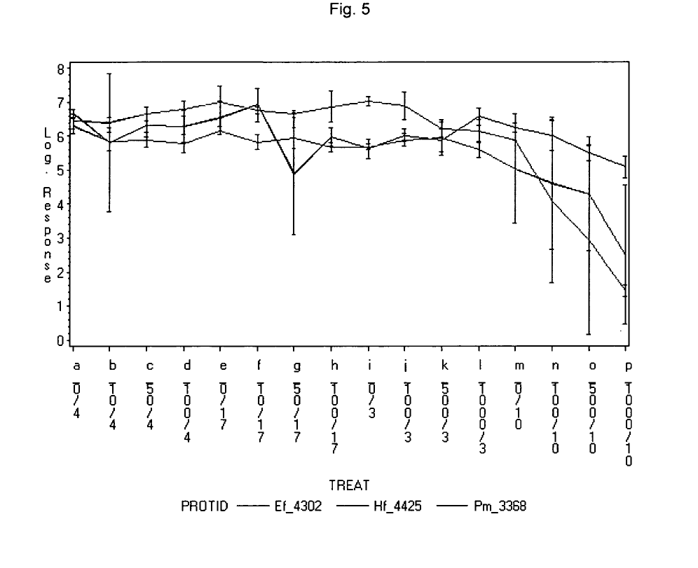

By contrast, the intersections in RUN2 are not empty, either in the binary or in the ternary intersection, and the ternary intersection deserves particular attention. This is because further examination shows that the protein identified in this intersection, namely L-FABP (PHEN_M_ID=3368, ETHI_F_ID=4302, HEXA_F_ID=4425) disappears after 10 d under the influence of treatment, and this effect is observed consistently in all three assays. FIG. 5 is a plot of the average treatment contrasts of the three assays over all 16 treatments and impressively demonstrates suppression of the protein by several powers of ten.

The protein L-FABP (ETHI_F=4302) shows by far the greatest correspondence of the influences of treatment, as shown by the pairwise correlation coefficients of the assays. With this protein, the average influences of treatment are virtually in agreement for the PHEN_M and HEXA_F assays, whereas there is a lower, but significant positive correlation of the PHEN_M, ETHI_F and HEXA_F, ETHI_F pairs.

The L-FABP proteins ETHI_F_ID 4302 and ETHI_F_ID 4316 are identified in three different preparative 2D gels. The livers of two female and one male rat are used for this. The protein spots are cut out, cleaved enzymatically in the gel with trypsin, and analyzed by the Nano-LC-MS/MS.

All six proteins were identified on the basis of their peptide masses and internal sequence tags as rat liver fatty acid binding protein (L-FABP, SWISS-Prot-ID P02692).

### 6.2. Western Blotting

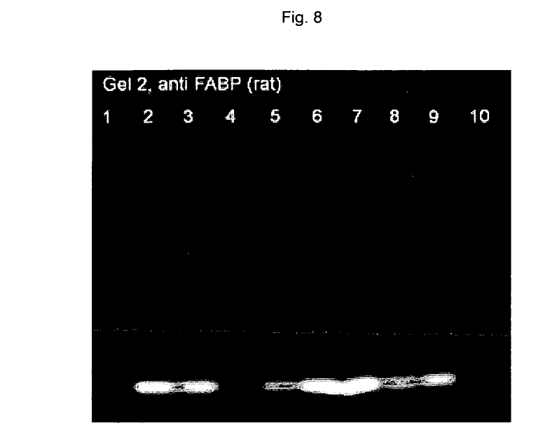

It was possible to confirm the results of the proteomic analysis for the substances phenobarbital, ethinylestradiol and alpha-hexachlorocyclohexane by Western blotting (FIGS. 7 and 8).

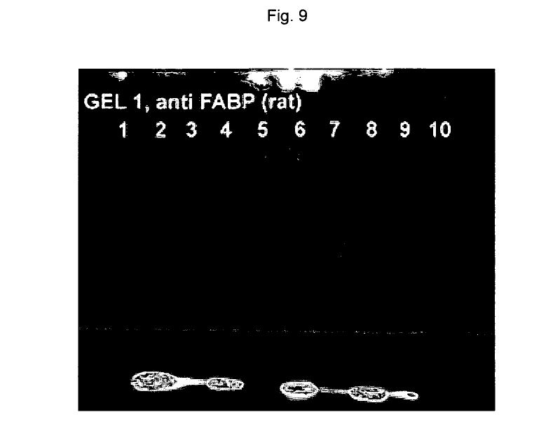

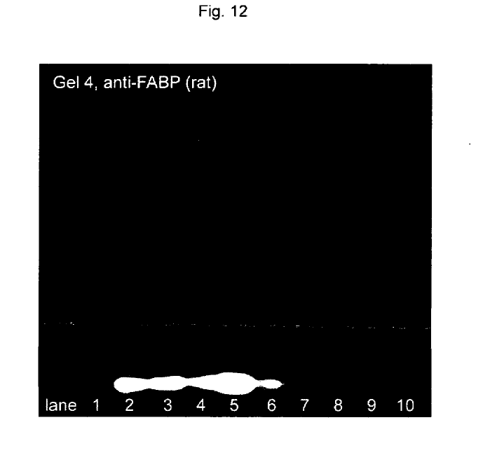

A decrease in L-FABP was likewise detected by Western blotting with the following compounds: tetrachloromethane, furan, 2,6-dinitrotoluene and cyproterone acetate (FIGS. 9, 10 and 12).

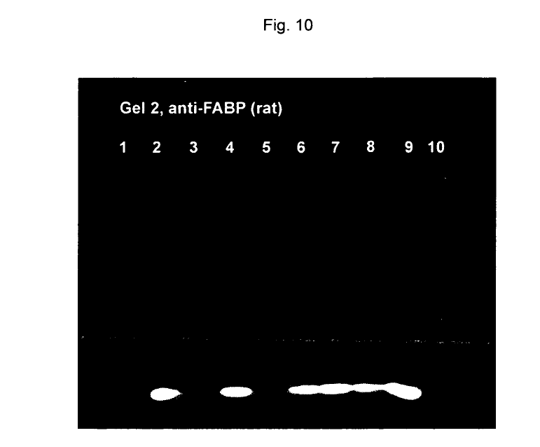

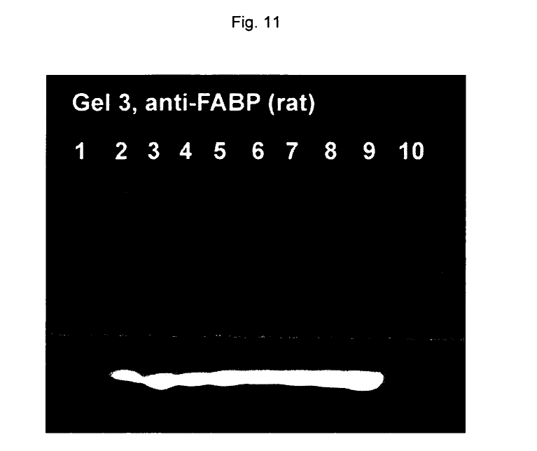

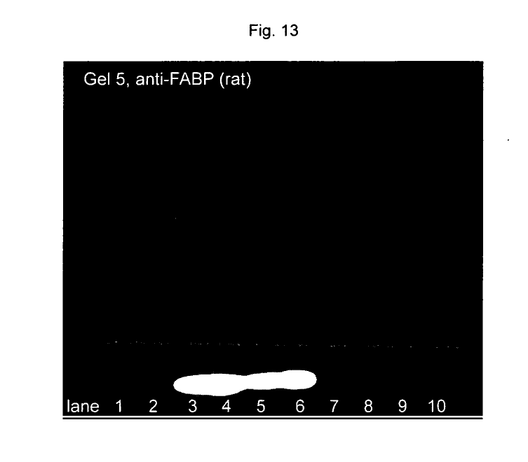

By contrast, no change in the L-FABP level resulted with the highest dosage chosen for 2,4-dinitrotoluene, 2,6-diaminotoluene, 2,4-diaminotoluene and with the two peroxisome proliferators WY 14,643 and nafenopin there was a tendency to a slight increase (FIGS. 10, 11, 12 and 13).

### 7. Discussion

The statistical analysis shows that there is significantly less expression in the liver of L-FABP (ETHI_F_ID 4302) in all investigated rats treated with synthetic estrogen ethinylestradiol and the two enzyme inducers phenobarbital and alpha-hexachlorocyclohexane, with longer exposure time and higher dosage, than in the controls. The protein spot ETHI_F_ID 4316 is located in the direct vicinity of ETHI_F_ID 4302 and shows an analogous profile of treatment effects to ETHI_F_ID 4392 in the graphical exploration, i.e. disappears after 10 days under the influence of treatment. The protein ETHI_F_ID 4302 does not differ in sequence from ETHI_F_ID 4316 and appears in the 2D gel at the same isoelectric point and at higher mass. This suggests a possible post-translational modification which has no effect on the pH. The difference between the two proteins might be a different N-glycosylation on asparagine (N) in the hexapeptide MEGDNK, which presumably for this reason was undetectable even once in the mass spectrometric peptide maps after tryptic cleavage of the protein.

It is of interest that expression of ETHI_F_ID 4302 was reduced highly significantly by several orders of magnitude on exposure to all three substances, dose-dependently, compared with the controls. An important observation in this connection is that the test substances used belong to different classes of mechanism of action, namely to the family of enzyme inducers and to substances having an estrogenic effect. Thus, L-FABP complies with several essential features for a marker significantly associated with tumor promotion:
Extended substance class correlation (enzyme induction and estrogenic effect)
Gender-independent: detection in male (phenobarbital) and female animals (ethinylestradiol and alpha-hexachlorocyclohexane (additional substances for female animals in the Western blot).
In addition, it is an early marker, the amount of which can be seen to be changed in the liver after only a few hours of exposure to the substances in the male animals treated with phenobarbital and the females treated with alpha-hexachlorocyclohexane.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method for testing substances or substance mixtures wherein
a) an organism or a part thereof is exposed to the substance or to the substance mixture, and
b) the expression of at least one liver fatty acid binding protein (L-FABP) is determined in at least one sample derived from the organism or the part, wherein the exposure time is more than 48 hours and a decrease in L-FABP expression brought about by exposure to the substance or to the substance mixture represents a toxic property of the substance or the substance mixture.

22. The method of claim 21 wherein the exposure time is 60 hours or more.

23. The method of claim 21 wherein the exposure time is up to 10 days.

24. The method of claim 21 wherein the exposure time is up to 5 days.

25. The method of claim 21 wherein the exposure time is up to 72 hours.

26. The method of claim 21 wherein the expression of at least one liver fatty acid binding protein (L-FABP) is determined before and after exposure to the substance or to the substance mixture.

27. The method of claim 26 wherein the L-FABP expression determined before and after exposure to the substance or to the substance mixture is compared with one another.

28. The method of claim 21 wherein the expression of at least one liver fatty acid binding protein (L-FABP) is determined for at least two different exposure times.

29. The method of claim 28 wherein
a1) a first organism or a part thereof is exposed to the substance or to the substance mixture;
b1) the expression of at least one liver fatty acid binding protein (L-FABP) is determined in at least one sample derived from the first organism or the part;
a2) a second organism or a part thereof is exposed to the substance or to the substance mixture; and
b2) the expression of the liver fatty acid binding protein (L-FABP) is determined in at least one sample derived from the second organism or the part, wherein the exposure time of the first organism or the part thereof is different from the exposure time of the second organism or the part thereof.

30. The method of claim 28 wherein
a) an organism or a part thereof is exposed to the substance or to the substance mixture; and
b) the expression of at least one liver fatty acid binding protein (L-FABP) is determined in at least a first and at least a second sample derived from the organism or the part,
wherein the first sample has been taken from the organism or the part thereof after a first exposure time and the second sample has been taken from the organism or the part thereof after a second exposure time, and the first exposure time is different from the second exposure time.

31. The method of claim 21 wherein the determination is carried out by protein analysis.

32. The method of claim 31 wherein the protein analysis is an immunological method.

33. The method of claim 31 wherein the protein analysis is a spectroscopic method.

34. The method of claim 21 wherein the organism is a rat.

35. The method of claim 21, wherein the part of the organism is a liver or a part thereof.

36. A method for identifying toxic properties of a substance or of a substance mixture comprising the method of claim 21.

37. The toxic properties of claim 36 wherein at least one of the properties is a tumor-promoting property.

38. The at least one tumor-promoting property of claim 37 selected from the group consisting of a receptor-mediated property, an enzyme-inducing property, a cytotoxic-mitogenic property, an oxidative stress-promoting property, and a mitochondrially toxic property.


SEQUENCE LISTING (1 sequence)

SEQ ID NO: 1 - protein, 127 aa, Rattus norvegicus
MNFSGKYQVQSQENFEPFMKAMGLPEDLIQKGKDIKGVSEIVHEGKKVKLTITYGSKVIHNEFTLGEECELETMTGEKVKAVVKMEGDNKMVTTFKGIKSVTEFNGDTITNTMTLGDIVYKRVSKRI